(19) United States
(12) Patent Application Publication
(10) Pub. No.: US 2023/0095800 A1
(43) Pub. Date: Mar. 30, 2023

(54) PULMONARY NEUROMUSCULAR METRIC DEVICE

(71) Applicant: BeCare Link LLC, Rumson, NJ (US)

(72) Inventors: LAWRENCE D. RUBIN, Rumson, NJ (US); MICHAEL A. HALEM, New York, NY (US)

(21) Appl. No.: 17/944,512

(22) Filed: Sep. 14, 2022

Related U.S. Application Data
(60) Provisional application No. 63/249,175, filed on Sep. 28, 2021.

(51) Int. Cl. A61B 5/085; A61B 5/097; A61B 5/087; A61B 5/00

(52) U.S. Cl. CPC A61B 5/085; A61B 5/097; A61B 5/0878; A61B 5/0002; A61B 5/742

(57) ABSTRACT

A pulmonary neuromuscular metrics device that allows the measurement of pulmonary neuromuscular metrics, that is, breathing power, force, and work (that is, energy expended), in patients with neuromuscular conditions. The device may be used in the medical office or remotely at a patient's house, thereby allowing the patient to be followed medically without the need for more frequent and repeated office visits.

### CROSS REFERENCE TO RELATED APPLICATION

This application claims the benefit of U.S. Provisional Application No. 63/249,175 filed on Sep. 28, 2021.

### FIELD OF THE INVENTION

Generally, the present disclosure relates to a device to measure, that is to produce metrics of, Pulmonary Neuromuscular Function in Patients with neuromuscular disorders.

### BACKGROUND

The prior art of testing Pulmonary Neuromuscular Function by use of spirometry is described in the standards of the American Thoracic Society: https://www.atsjournals.org/doi/10.1164/rccm.201908-1590ST

There are many neuromuscular disorders which affect the Neuromuscular Pulmonary Function of patients. The pulmonary neuromuscular metrics of each such patient needs to be tracked with time to care for these patients. The goal of the instant invention is to facilitate such tracking of such metrics for such patients.

In the present disclosure, where a document, an act and/or an item of knowledge is referred to and/or discussed, then such reference and/or discussion is not an admission that the document, the act and/or the item of knowledge and/or any combination thereof was at the priority date, publicly available, known to the public, part of common general knowledge and/or otherwise constitutes prior art under the applicable statutory provisions; and/or is known to be relevant to an attempt to solve any problem with which the present disclosure may be concerned with.

### SUMMARY OF THE INVENTION

The instant invention is novel over the prior art use of spirometry for a number of reasons as discussed herein, one of which being that this invention, unlike spirometry, is based on using a resistance device (in one case a metering orifice at the end of a breathing tube) that causes the patient to work against the resistance, thereby measuring the patient's neuromuscular strength and fitness which may markedly decline as an illness progresses.

The present disclosure addresses the above problems, and may also prove useful in addressing other problems and deficiencies in a number of other medical areas. Therefore, the claims, as recited below, should not necessarily be construed as limited to addressing any of the particular problems or deficiencies discussed herein.

The following definitions will be used in the present disclosure for the convenience of the reader:

“Pulmonary Neuromuscular Function” is measurement of the impact of the health of nerves and muscles on respiration.

“Patient” is a human being or animal, whether in a physician's or other medical or veterinarian care professional's office, in a location remote to the professional's office including the home, in a laboratory, at a farm, or any other place where the Patient's metrics are being taken for the purpose of medical care.

“Medical Care” is medical or veterinarian care for a Patient, and includes self-care and therapy.

“Medical Professional” is a physician, nurse, doctor, veterinarian, assistant, therapist, or any other person who administers Medical Care to a Patient.

“Pulmonary Neuromuscular Metrics” are any metrics or measurements taken of Pulmonary Neuromuscular Function that can be affected by any neuromuscular disease.

“Traditional Pulmonary Metrics” are those metrics previously measured by spirometry including flow, volume, and static pressure or suction taken by the Patient.

“New Pulmonary Neuromuscular Metrics” are those metrics specifically introduced for the first time with a Patent being diagnosed or for a Patient's prior diagnosis being continued based on the Patient's ability to generate dynamic flow through an orifice, dynamic power through an orifice, or dynamic energy expended through an orifice.

“Therapeutic Pulmonary Exercises” are strengthening or endurance exercises specifically using the resistance of a Metering Orifice which are performed with the intention of respiratory and/or neuromuscular therapy for the patient to strengthen, increase endurance, remove obstructions, remove secretions, lower respiratory resistance, train neuromuscular function, train neuromuscular coordination, train neuromuscular proprioception, or otherwise improve the respiration of the Patient.

“Pulmonary Neuromuscular Metric Device” or “the Device” is an electronic device of the instant invention that allows for the measurement of New Pulmonary Neuromuscular Metrics and may also allow for the measurement of certain Traditional Pulmonary Metrics.

“Metering Tube” is a tube, cylinder, sphere, ovoid spheroid, or any other shaped hollow volume, vessel, or construct having: an opening at one end to connect to the mouth, nose, or nose and mouth via a breathing mask or mouthpiece, of a Patient; a “Metering Restriction”, i.e. a metering hole, exit tube, porous filter, orifice, or any other restriction which provides back pressure against the flow of a gas across the restrictions; and at least one port in the wall of said tube, cylinder, sphere, ovoid sphere, or any similar hollow volume, vessel, or construct located between the ends of said tube for connection to one or more sensors or small pipe connections for one or more sensors to measure physical properties of the gas inside the metering tube including pressure, and in some embodiments temperature, humidity gas composition, and such other physical properties as needed to compute density, volume, and resistance to movement through the Metering Restriction.

“Device Electronics Package” is an electronic device in some embodiments containing one or more ports, one or more sensors, electronics, at least one CPU, a memory, data storage devices, batteries, displays, speakers, microphones, and external digital communications. The Device Electronics Package may be a single unit or a collection of separate units connected electronically, that is, by physical connections or wireless connections, within Pulmonary Neuromuscular Metering Device.

As taught in the instant invention, the Device consists of the Metering Tube which may be a hollow volume of any shape that is convenient to the placement of the sensors and is biomechanically comfortable for a Patient. The preferred embodiment of said Device is comprised of a Metering Tube as a hollow cylinder. A Patient places his or her mouth at the proximal end of the Metering Tube. A Metering Restriction is located on the Metering Tube, be it on the end or somewhere else on its surface (i.e. half-way down), which is a calibrated orifice, that serves as an resistance to the movement of air from the inside of the Metering Tube to the outside, or back, depending on whether the Patient is exhaling or inhaling. In the preferred embodiment, a smaller perpendicular tube is located approximately one half of the way down the Metering Tube from the proximal end, which tube serves as a connection to an air-pressure sensor, which may measure absolute pressure or differential (with respect to ambient air) pressure contained within the Device Electronics Package. Said sensor itself may be electronics located within the Metering Tube and contain additional sensor types (i.e. temperature, humidity, CO2), or it may be connected via another tube to an external sensor located within the Device Electronics Package.

The Device Electronics Package contains all sensors that are not mounted in the Metering Tube, one or more controls to for the patient to operate the device (such as, an on/off button), and a digital computer (“CPU”) to process the sensor data and either store the data in the Device, display the data on the Device itself, and/or transmit the data to a Medical Professional.

Said data may be transmitted securely via a wireless connection, such as WiFi or Bluetooth, to the cloud, or via a wireless or by a wired connection to a local computer, tablet, or cell phone. The data may be displayed locally, or in the preferred embodiment, may be transmitted to be stored in the cloud, in which Pulmonary Neuromuscular Metrics are curated and collated for the Medical Professional or team reviewing the status of the Patient.

### BRIEF DESCRIPTION OF THE DRAWINGS

FIG. 1 is a block diagram of the preferred embodiment of the system of the instant invention described herein.

FIG. 2 is a flowchart of the device start up segment of the application program of the instant invention.

FIG. 3 is a flowchart of the run-loop segment of the application program of the instant invention.

FIG. 4 is a flowchart of the data transfer segment of the application program of the instant invention.

FIGS. 5, 6, 7, and 8 taken together are a flowchart of the data analysis segment of the application program of the instant invention.

The accompanying drawings illustrate example embodiments of the present disclosure. Such drawings are not to be construed as necessarily limiting the present disclosure. Like numbers and/or similar numbering scheme can refer to like and/or similar elements throughout. All example embodiments are mere example and the invention is not limited to any such embodiment, but rather encompasses all embodiments as explained in the “Detailed Description of the Invention below.

### DETAILED DESCRIPTION OF THE INVENTION

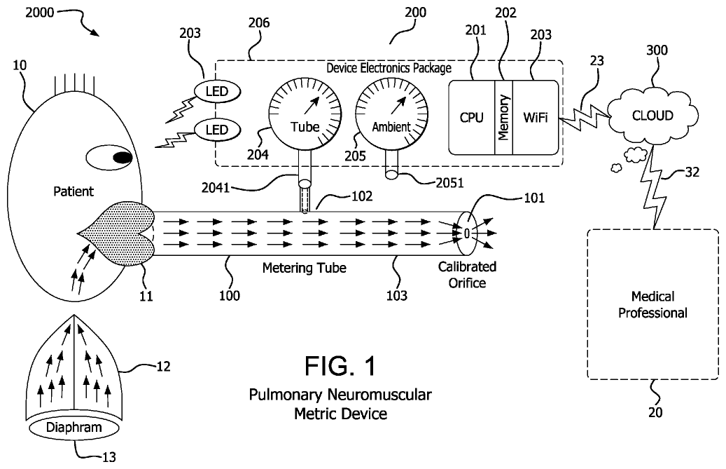

The preferred embodiment of the system 2000 of the instant invention is shown in FIG. 1. A Patient 10 exhales or inhales by placing his or her lips 11 on the mouthpiece, a proximal input of a tube 100 with a metering restriction, hole, or calibrated orifice, 101 at its other, or distal, end. Tube 100 has a port 102 constructed in its wall 103 for access to the air inside tube 100 by a perpendicular tube 2041 that feeds sensor 204. Sensor 204 takes physical measurements of the pressure, and optionally temperature, humidity, and gas composition inside cylinder 100 as generated by air flow through the lungs 12 of Patient 10 powered by his or her diaphragm 13. A second sensor 205 takes physical measurements of the pressure, and optionally temperature, humidity, and gas composition, in the ambient air through a port 2051 that is constructed in the physical enclosure 206 of device 200. FIG. 1 shows two light emitting diodes (LEDs) that provide feedback in the preferred embodiment to Patient 10 as to the completion or status of the test; it should be noted that other types of displays, such as an electronic display screen, can be employed to provide such feedback. A Device Electronics Package 200 includes a central processing unit, or CPU, 201 running under the control of an application software program (the “Program”) that is stored in whole or in part in memory 202 (or in an alternate embodiment stored in whole or in part in a server that is connected to the cloud 300). In one embodiment, the system 2000 collects data from the sensor 204 accessing tube 100 and from Ambient sensor 205, and securely transmits the data collected by sensors 204 and sensors 205 that is processed by CPU 201 under control of the Program, which processed data is transmitted via wireless connection 23 to the Cloud 300 for transformation into the Pulmonary Neuromuscular Metrics that are made accessible to a Medical Professional 20 via wireless transmission 32.

Metering Tube 100 in the preferred embodiment is a tube or cylinder with an opening at its proximal end to connect to the mouth, nose, or nose and mouth via a breathing mask or mouthpiece of a Patient 10. Metering Tube 100 at its distal end comprises a Metering Restriction or calibrated orifice 101 that provides back pressure against the flow of a gas across the restrictions and has one or more longitudinally placed ports for access by small approximately perpendicularly placed pipe or tube connections 2041 for one or more sensors 204 to measure physical properties of the gas inside said metering tube 100, including pressure, and optionally temperature, humidity gas composition, and such other physical properties as needed to compute density, volume, and resistance to movement of the forced air through the Metering Restriction 101.

FIG. 1 shows a Device Electronics Package 200 in which are resident digital electronic sensors 204 and 205 connected respectively to perpendicular tube 2041 and port 2051, a CPU 201, and a memory 202. Said package 200 may include in certain embodiments data storage devices, batteries, connections to power supplies, displays, speakers, microphones, or external digital communication hardware and software.

Device Electronics Package 200 gathers data from sensors 204 and 205 and may, in certain embodiments, store said data in memory 202 , or may display said data on a display screen of said package 200 itself, or may transmit said data to said cloud 300 for processing by a server connected to said cloud 300, and eventually sent to or accessed by a Medical Professional 20 for review, whether by reading graphs or tabulated data, and whether on a smartphone, a table, a laptop, or on a display resident in or on said Device Electronics Package 200 or connected to said Device Electronics Package by wireless or wired connection. Said data may be transmitted securely via a wireless connection 23, such as WiFi or Bluetooth, to said cloud 300 for processing or storage, or may be wireless connected or physically wired to directly a local computer, tablet, or cell phone for processing or storage. The data may be displayed locally, or in the preferred embodiment, may be transmitted to be stored in said cloud 300, in which Pulmonary Neuromuscular Metrics may be curated or collated for Medical Professional 20 or team of medical professionals reviewing the status of said Patient 10. A minimal embodiment of the present invention may contain only a pressure sensor 204 connected to said Tube 100; however, the preferred embodiment employs sufficient additional sensors for ambient (e.g. room or other conditions outside said Tube 100) and inside the Metering Tube 100 air temperature and pressure, humidity, and optionally CO2 concentration so that more accurate calculations can be made. A simplified embodiment may also be serviceable using solely a differential pressure sensor between ambient pressure and pressure inside the Tube 100.

Other embodiments may or may not use one or all additional sensors for, without limitation: (a) ambient temperature inside said Tube 100; (b) ambient pressure, inside Tube 100; (c) ambient humidity inside Tube 100; (d) ambient CO2 concentration inside Tube 100; (e) CO2 concentration inside the Tube 100; (f) ambient air density, inside Tube 100 air density; (g) ambient air specific heat at constant volume inside Tube 100; (h) gamma (the ratio of specific heat at constant pressure over constant volume) of ambient air; and (i) gamma of air inside the Tube 100.

In cases in which one or more sensor measurements are not available, the missing values may be estimated from such sources including the national weather service, tables, formulae, rules of thumb, text books, encyclopedias, Wikipedia, the internet, and publish papers; and also as examples standard temperature and pressure, sea level standard pressure, typical room temperature, weather service reported temperature, pressure, and humidity at sites near the address of said Patient 10, the altitude of dwelling place of said Patient 10 or the office of said Medical Professional 20, standard body temperature, the saturation of air at 100% humidity, the constituent gases of air at the surface of earth, typical humidity, temperature, and pressure of air-conditioned or heated rooms, typical CO2 concentrations in dwelling rooms, typical CO2 concentrations in exhaled air, and such other reasonable basis for estimation of the physical properties of ambient air and air inside said Tube 100, including density and gamma

In the case in which said Calibrated Resistance 101 is not in the form of an orifice, but is rather a different resistance means (i.e. a filter), the word “orifice” can be replaced with “resistance” in FIG. 1. The use of a resistance against dynamic flow is a point of novelty disclosed by this invention.

Using the Program that includes an algorithm (the “Algorithm”) that uses calibration and gas law physics for translating sensor readings of the Device, the following measurements of Pulmonary Neuromuscular Metrics can be determined for transmission to a Medical Professional 20: Tidal Volume (TV); Inspiratory Reserve Volume (IRV); Expiratory Reserve Volume (ERV); Inspiratory Capacity (IC); Vital Capacity (VC); Max Inspiratory Pressure Against a Calibrated Orifice (MIPCO); Max Expiratory Pressure Against a Calibrated Orifice (MEPCO); Power of Breathing Against a Calibrated Orifice (POWCO); Max Inspiratory Power of Breathing Against a Calibrated Orifice (MIPOWCO); Max Expiratory Power of Breathing Against a Calibrated Orifice (MEPOWCO); Average Sustained Inspiratory Power of Breathing Against a Calibrated Orifice (SIPOWCO) [sequential best inspirations]; Average Sustained Expiratory Power of Breathing Against a Calibrated Orifice (SEPOWCO) [sequential best expirations]; Average Sustained Tidal Inspiratory/Expiratory power (TIPOWCO and TEPOWCO); Average Sustained Tidal Inspiratory/Expiratory power (TIPOWCO and TEPOWCO); Average Sustained Work of Breathing per Liter Against a Calibrated Orifice (WOBCO); Minute Ventilation (MINUTEV); and Cumulative Energy Expended forcing air through Resistance (CEEFR). It should be noted that all instantaneous, average, and peak metrics can be displayed in a graphic display against time, for example, in one embodiment a graph of Power of Breathing Against a Calibrated Orifice (POWCO) on the y axis and time on the x axis. Said system 2000 is capable of using additional sensor data is collected for additional accuracy in computing Pulmonary Neuromuscular Metrics, including any of: a) differential pressure between the metering tube and the room (ambient); b) ambient (room) pressure; c) temperature in Metering Tube 100; d) ambient temperature; e) humidity in Metering Tube 100; f) ambient humidity; g) CO2 concentration in Metering Tube 100; h) ambient CO2 concentration; h) O2 in Metering Tube 100; or i) ambient O2.

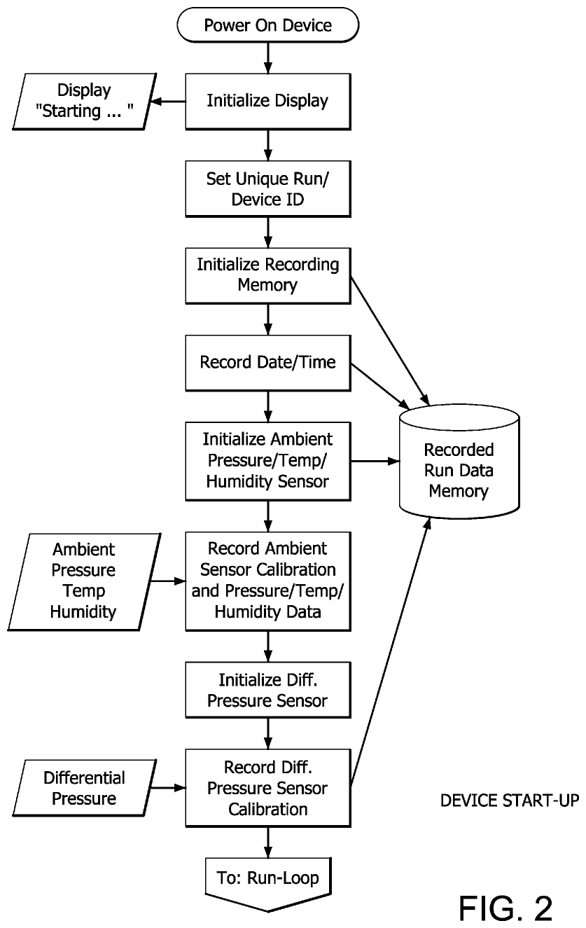

FIG. 2 is a flowchart that sets out the steps of a subroutine of said Program that controls start up of system 2000 of the preferred embodiment having a display screen in or connected to Device 200 visible to Patient 10 or Medical Professional 20 using said system 2000. In the preferred embodiment, the steps in FIG. 2 are carried out by CPU 201 running the Program stored in memory 202.

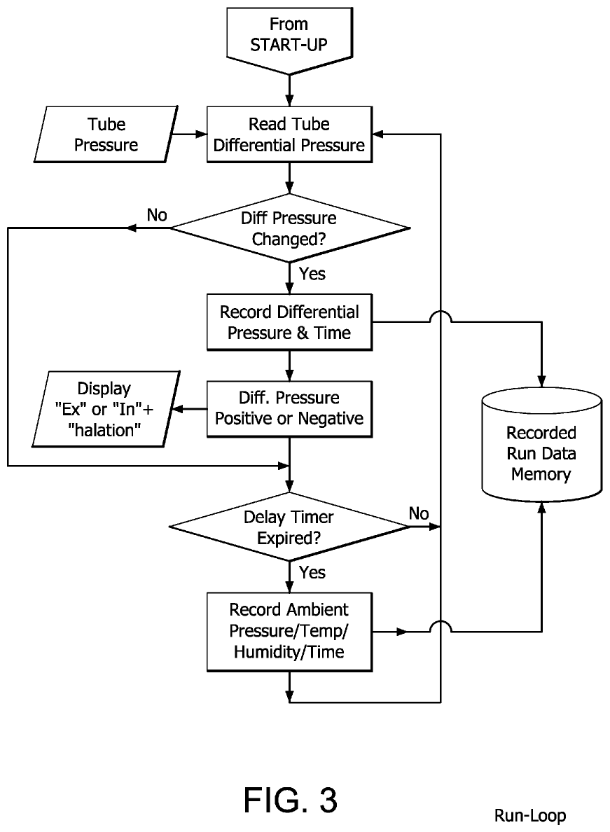

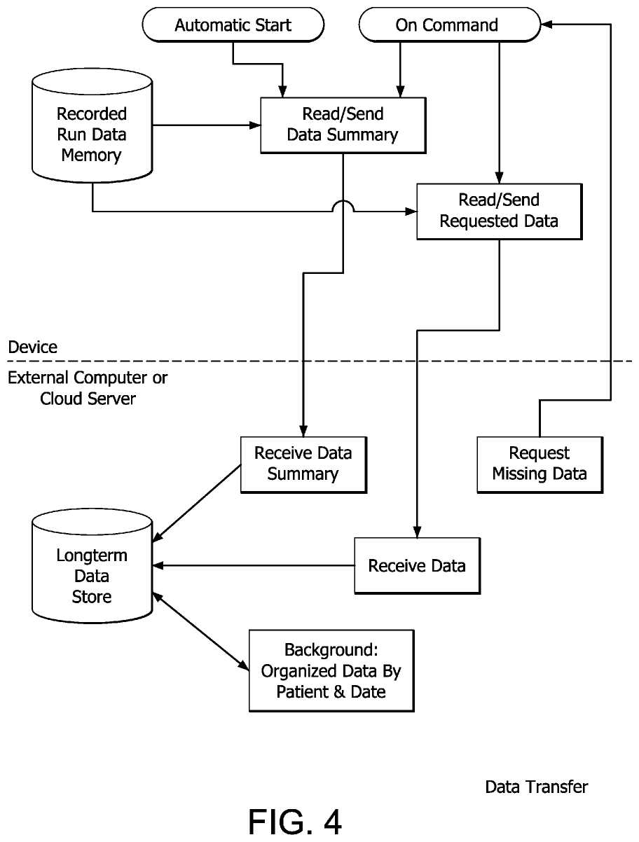

FIG. 3 is a flowchart that sets out the steps of a subroutine of the Program stored in memory 202 under control of CPU 201 providing for the running of the Device Electronics Package 200 of the preferred embodiment. In the preferred embodiment, the steps of the subroutine of the Program as set forth in the flowchart of FIG. 4 are carried out under the control of CPU 201 for the collection of data collected by Device Electronics Package 200. Said data is stored in memory 202 and then transferred under control of CPU 201 to an external computer or cloud 300 server for processing, storage, and transfer to a medical professional 20 for review.

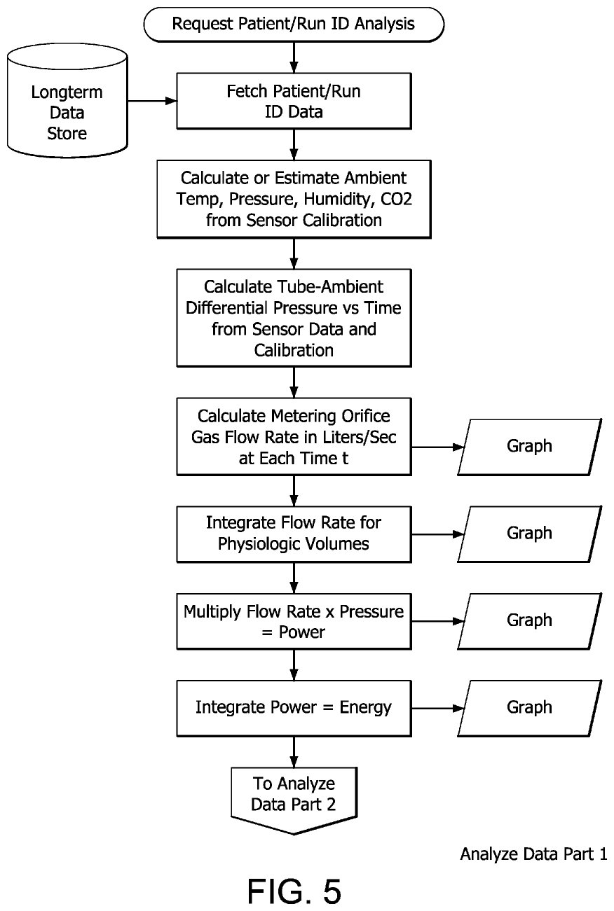

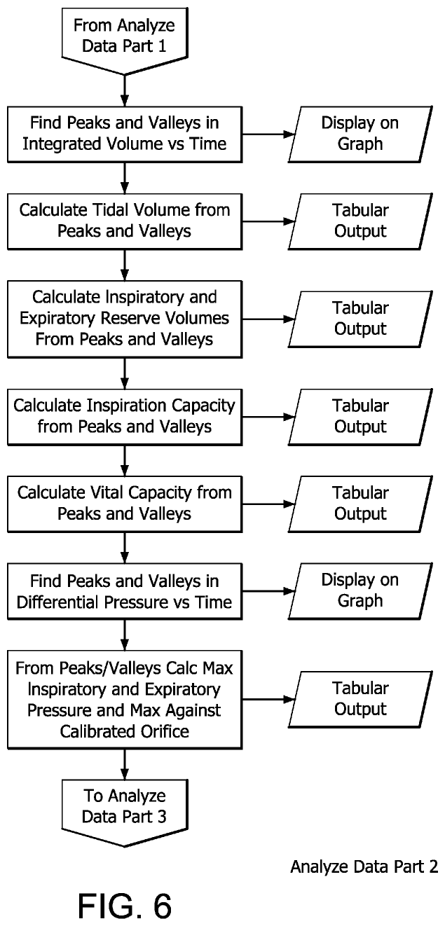

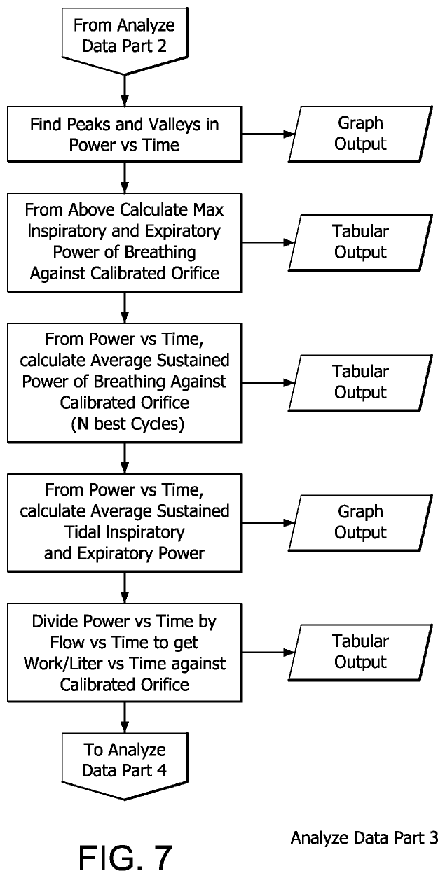

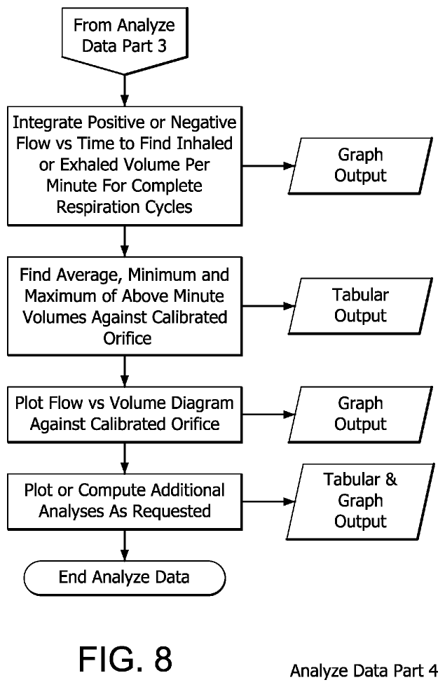

Taken together, the flowcharts of FIG. 5, FIG. 6, FIG. 7, and FIG. 8 set out the steps of a subroutine of the Program that control the analysis of data gathered and processed by CPU 201 of the Device Electronics Package 200; in one embodiment said steps may be performed within Device Electronics Package 200, or in another embodiment, by a cloud 300 server or external computer. While it should be appreciated that the primary use of the device 200 of the instant invention is to measure (that is, to produce metrics of Pulmonary Neuromuscular Function for the purpose of the diagnosis, either initial or ongoing, and treatment of patients with neuromuscular disorders, it is not intended that this application be read narrowly so as to limit potential additional uses of said Device that may be covered by claims directed to such uses. Such other uses shall include, but not be limited to, the use of the system 2000 by a Patient 10 to perform Therapeutic Pulmonary Exercises.

What is claimed is:

1. A system for measuring pulmonary neuromuscular metrics of a patient comprising:
a hollow volume having two ends with a mouthpiece constructed at one end, a resistance at the opposite end, and a port in the wall of said hollow volume located between said ends; and
an electronic device having a first sensor, said sensor connected to said port in said wall,
whereby pulmonary neuromuscular metrics for said patient exhaling or inhaling at said mouthpiece are provided for reading by said patient's medical professional.

2. The system of claim 1 in which said hollow volume is selected from the group comprising: a tube, a cylinder, a sphere, and an ovoid spheroid.

3. The system of claim 1 in which said sensor measures air pressure.

4. The system of claim 3 in which said sensor measures air temperature.

5. The system of claim 1 in which said mouthpiece is selected from the group comprising: an opening for connection to the mouth of said patient; an opening for connection to the nose of said patient; an opening for connection to the mouth and the nose of said patient; and a breathing mask.

6. The system of claim 3 in which the output of said sensor is data relating to pulmonary neuromuscular metrics.

7. The system of claim 4 in which the output of said sensor is data relating to pulmonary neuromuscular metrics.

8. The system of claim 1 in which said device additionally comprises:
a port;
a second sensor;
a central processing unit;
a power supply;
a memory, and
a wireless transmitter,
whereby said second sensor has access to ambient air through said port.

9. The system of claim 8 in which said memory has resident therein an application program.

10. The system of claim 9 in which said application program is comprised of a start up subroutine, a run-loop subroutine, a data transfer subroutine, a data analysis subroutine, and an algorithm for translating sensor readings of said first sensor and said second sensor into pulmonary neuromuscular metrics.

11. The system of claim 1 in which said pulmonary neuromuscular metrics are selected from a group comprising: tidal volume (TV); inspiratory reserve volume (IRV); expiratory reserve volume (ERV); inspiratory capacity (IC); vital capacity (VC); max inspiratory pressure against a calibrated orifice (MIPCO); max expiratory pressure against a calibrated orifice (MEPCO); power of breathing against a calibrated orifice (POWCO) max inspiratory power of breathing against a calibrated orifice (MIPOWCO); max expiratory power of breathing against a calibrated orifice (MEPOWCO); average sustained inspiratory power of breathing against a calibrated orifice (SIPOWCO); average sustained expiratory power of breathing against a calibrated orifice (SEPOWCO); average sustained tidal inspiratory power (TIPOWCO); average sustained tidal expiratory power (TEPOWCO); average sustained work of breathing per liter against a calibrated orifice (WOBCO); minute ventilation (MINUTEV); and cumulative energy expended forcing air through resistance (CEEFR).

12. The system of claim 8 in which said device additionally comprises at least one light emitting diode.

13. The system of claim 8 in which said device additionally comprises a display screen.

14. The system of claim 13 in which said pulmonary neuromuscular metrics are mapped graphically on said display screen as a function of time.

15. The system of claim 6 in which said data is transmitted wirelessly to a secure computer cloud server running an algorithm that generates said pulmonary neuromuscular metrics.

16. The system of claim 7 in which said data is transmitted wirelessly to a secure computer cloud server running an algorithm that generates said pulmonary neuromuscular metrics.

17. The system of claim 1 in which said pulmonary neuromuscular metrics are transmitted to a computing device having a display screen in the possession of said medical professional.

18. The system of claim 1 further comprising at least one additional sensor selected from the group comprising: an air pressure sensor; an air temperature sensor; a humidity sensor; a carbon dioxide sensor; and an oxygen sensor.

19. The system of claim 8 further comprising at least one additional sensor selected from the group comprising: an air pressure sensor; an air temperature sensor; a humidity sensor; a carbon dioxide sensor; and an oxygen sensor.

20. A system for use by a patient in performing therapeutic pulmonary exercises comprising:
a hollow volume having a mouthpiece, a resistance to air flow, and a port in the wall of said hollow volume located between said mouthpiece and said resistance; and
an electronic device having at least one sensor and a display screen, said sensor connected to said port in said wall of said hollow volume,
whereby said patient is prompted by directions displayed on said display screen for performing therapeutic pulmonary exercises by exhaling or inhaling at said mouthpiece with the result that pulmonary neuromuscular metrics for said patient exhaling or inhaling at said mouthpiece are provided for reading by said patient's medical professional.

21. A method for measuring pulmonary neuromuscular metrics for a patient comprising the steps of:
powering on an electronic device;
initializing an ambient air pressure sensor resident in said electronic device having access to ambient air;
initializing an air pressure sensor resident in said electronic device having access to air in a hollow volume having a mouthpiece at a proximal end;
exhaling by said patient into said mouthpiece;
measuring the differential air pressure between the ambient air pressure and the air pressure in said hollow volume, said tube having a resistance to air flow at a distal end;
recording said differential air pressure by said electronic device;
storing said recording in a memory;
analyzing said stored recordings using an algorithm,
storing said analysis in a memory; and
displaying said analysis
whereby pulmonary neuromuscular metrics for said patient are provided for reading by said patient's medical professional.
